(19) United States
(12) Patent Application Publication
(10) Pub. No.: US 2022/0341945 A1
(43) Pub. Date: Oct. 27, 2022

(54) Biomarker for diagnosing depression and uses thereof

(71) Applicant: EULJI UNIVERSITY INDUSTRY ACADEMIC COOPERTAION FOUNDATION, Gyeonggi-do (KR)

(72) Inventors: Hee-Gyoo KANG, Seoul (KR); Jiyeong LEE, Gyeonggi-do (KR); Hyebin CHOI, Gyeonggi-do (KR); Eun-Jeong JOO, Seoul (KR)

(21) Appl. No.: 17/387,483

(22) Filed: Jul. 28, 2021

(30) Foreign Application Priority Data
Apr. 23, 2021 (KR) 10-2021-0053114

(51) Int. Cl. G01N 33/68

(52) U.S. Cl. CPC G01N 33/6893; G01N 2333/4742; G01N 2800/304

(57) ABSTRACT

The present invention relates to a marker composition for diagnosing major depressive disorder, comprising ZA2G and prothrombin as markers, a method for providing information necessary to determine the occurrence of major depressive disorder using the marker composition, a composition for determining the occurrence of major depressive disorder, comprising agents for measurement of the expression levels of the markers, and a kit for determining the occurrence of major depressive disorder, comprising devices for measurement of the expression levels of the markers. The method for providing information for use in determining the occurrence of major depressive disorder provided by the present invention can be widely utilized to determine the occurrence of various mental disorders, including major depressive disorder since it is possible to measure the expression levels of proteins of which the expression levels are changed at the time of the occurrence of major depressive disorder, and to more objectively and accurately determine the occurrence of major depressive disorder when the method is used.

### TECHNICAL FIELD

The present invention relates to a biomarker for diagnosing major depressive disorder and uses thereof. More specifically, the present invention relates to a marker composition for diagnosing major depressive disorder, comprising ZA2G and prothrombin as markers, a method for providing information necessary to determine the occurrence of major depressive disorder using the marker composition, a composition for determining the occurrence of major depressive disorder, comprising agents for measurement of the expression levels of the markers, and a kit for determining the occurrence of major depressive disorder, comprising devices for measurement of the expression levels of the markers.

### BACKGROUND ART

Major depressive disorder (MDD) is a mental disorder that affects more than 300 million people worldwide, and causes the greatest burden on mankind. Patients with MDD can resume work before an average of 70 days when adequate treatment for MDD is provided, and a remission rate of 76% is acquired when patients with MDD are provided with adequate treatment. However, since there are no biomarkers or imaging diagnostic methods that can be used to diagnose MDD, there is difficulty in diagnosing MDD.

MDD diagnosis methods currently used are in the form of questionnaires, counseling by a professional counselor, and drug treatment, and have been developed for the purpose of analyzing the condition of the subject and improving the MDD status. However, in the case of questionnaires, there is a problem in that the contents of questionnaires are not suitable for the surrounding environment, gender, age, and the like of the subject thereof. Accordingly, research to develop a method for diagnosing MDD has been actively conducted. For example, Korean Patent No. 10-2192345 discloses a technique for diagnosing MDD by analyzing brain waves based on frequency-resolution, and Korean Laid-Open Patent Application No. 10-2020-0061016 discloses a method for diagnosing MDD using facial skin images. However, the brain waves analysis method or the facial skin image is not actually used for diagnosing MDD since there is a problem in that the brain waves or the facial skin image cannot be quantified as an objective value because the deviation is severe depending on the condition of the patient.

With this background, the present inventors have made intensive research efforts to develop a method for diagnosing major depressive disorder more objectively, as a result, confirmed that the occurrence of major depressive disorder can be more objectively determined by detecting the expression levels of ZA2G and prothrombin contained in the serum and combining the expression levels, thereby completing the present invention.

### DISCLOSURE

### Technical Problem

A main object of the present invention is to provide a marker composition for diagnosing major depressive disorder, comprising a protein selected from the group consisting of ZA2G, prothrombin, and a combination thereof as a marker.

Another object of the present invention is to provide a method for providing information necessary to determine the occurrence of major depressive disorder using the marker composition.

Still another object of the present invention is to provide a composition for determining the occurrence of major depressive disorder, comprising an agent for measurement of the expression level of the marker.

Still another object of the present invention is to provide a kit for determining the occurrence of major depressive disorder, comprising a device for measurement of the expression level of the marker.

Still another object of the present invention is to provide the use of ZA2G and prothrombin to diagnose major depressive disorder.

### Technical Solution

Each description and embodiment disclosed in this disclosure may also be applied to other descriptions and embodiments. That is, all combinations of various elements disclosed in this disclosure fall within the scope of the present disclosure. Further, the scope of the present disclosure is not limited by the specific description below.

Further, those skilled in the art will recognize, or be able to ascertain using no more than routine experimentation, many equivalents to the specific embodiments of the invention described herein. Further, these equivalents should be interpreted to fall within the scope of the present invention.

In addition, throughout this specification, when a part is referred to as “including” an element, it will be understood that other elements may be further included rather than other elements being excluded unless content to the contrary is specially described.

Hereinafter, the present invention will be described in detail.

An embodiment of the present invention for achieving the objects provides a marker composition for diagnosing major depressive disorder, comprising a protein selected from the group consisting of ZA2G (zinc-alpha-2-glycoprotein), prothrombin, and a combination thereof as a marker.

In order to develop a method for objectively evaluating and determining the occurrence of major depressive disorder, the present inventors have conducted various studies to discover protein markers of which the expression levels change depending on the occurrence of major depressive disorder among proteins contained in the serum. As a result, the present inventors have confirmed that the expression levels of ZA2G and prothrombin change depending on the occurrence of major depressive disorder, and discovered ZA2G and prothrombin as markers.

As used herein, the term “major depressive disorder” does not mean a status in which only mood is temporarily lowered but means a status in which overall mental functions such as content of thoughts, thought processes, motivation, will, interest, behavior, sleep, and physical activity are lowered.

As used herein, the term “diagnosis” includes the act of determining the susceptibility of a subject to a particular disease or illness; the act of determining whether a subject currently has a particular disease or illness; the act of determining the prognosis of a subject having a particular disease or illness; or therametrics (for example, the act of monitoring the condition of a subject to provide information on treatment efficacy).

In the present invention, the diagnosis of major depressive disorder may be interpreted as meaning an act of objectively determining whether major depressive disorder has occurred in a targeted patient.

As used herein, the term “zinc-alpha-2-glycoprotein (ZA2G)” refers to a kind of soluble protein that stimulates lipolysis and induces body fat reduction in mice, and is known to be related to the occurrence of cachexia caused by cancer and mainly expressed in secretory cells of the lung epithelium. The specific amino acid sequence of ZA2G or the nucleotide sequence information of a gene encoding the same is reported in a database such as the NCBI. For example, the sequence is reported as GenBank Accession Nos. CCP80322.1, AAH05908.1, and the like.

As used herein, the term “prothrombin” refers to a blood coagulation-related protein that is circulated as prothrombin in an inactive state when being synthesized in the liver and secreted into the blood. It is known that prothrombin is converted into thrombin by the coagulation factor Xa/Va complex and calcium in a general mechanism of blood coagulation. The specific amino acid sequence of prothrombin or the nucleotide sequence information of a gene encoding the same is reported in a database such as the NCBI. For example, the sequence is reported as GenBank Accession Nos. NM_000506, NM_001311257, NM_010168, and the like.

Meanwhile, in addition to ZA2G (zinc-alpha-2-glycoprotein) and prothrombin, K2C1 (keratin type II, cytoskeletal 1) may be additionally used as a marker protein for diagnosis of major depressive disorder.

As used herein, the term “keratin type II, cytoskeletal 1 (K2C1)” refers to a member of the keratin family, also referred to as “keratin1”. Keratin is divided into two types of type 1 keratin that is acidic keratin and type 2 keratin that is basic keratin, and K2C1 belongs to type 2. The specific amino acid sequence of K2C1 or the nucleotide sequence information of a gene encoding the same is reported in a database such as the NCBI. For example, the sequence is reported as GenBank Accession Nos. NP_990263.1, KAB1275000.1, and the like.

Another embodiment of the present invention provides a method for providing information necessary to determine the occurrence of major depressive disorder using the marker composition.

Specifically, the method for providing information necessary to determine the occurrence of major depressive disorder according to the present invention includes quantitatively analyzing the expression level of a marker protein selected from the group consisting of ZA2G (zinc-alpha-2-glycoprotein), prothrombin, and a combination thereof in a serum sample of an individual suspected of having major depressive disorder.

As used herein, the term “individual” may include, without limitation, mammals including rats, livestock, and humans, farmed fish, and the like, which are likely to develop or have major depressive disorder.

As described above, K2C1 may be additionally used in addition to the ZA2G and prothrombin.

In the present invention, any method known to those skilled in the art may be used for the step of quantitatively analyzing the expression level of a protein. As a specific example, PCR, ligase chain reaction (LCR), transcription amplification, self-sustained sequence replication, nucleic acid sequence-based amplification (NASBA) method, and the like may be used, but the analysis method is not limited thereto. Here, the amino acid sequence of the marker protein for diagnosis of major depressive disorder according to the present invention or the nucleotide sequence of a gene encoding the same is known in a database such as the NCBI, and those skilled in the art may use an appropriate means required to measure the protein expression level.

As described above, the method may further comprise quantitatively analyzing the expression level of K2C1 (keratin type II, cytoskeletal 1) in addition to the ZA2G and prothrombin.

Meanwhile, the method may further comprise correlating the level of ZA2G (zinc-alpha-2-glycoprotein) or prothrombin, which is a protein quantitatively analyzed from the serum sample, with the determination of the occurrence of major depressive disorder.

In other words, since there is a deviation in the quantitatively analyzed level of each protein depending on the condition of the patient, it is not easy to use only the quantitatively analyzed fragmentary level of a protein to determine the occurrence of major depressive disorder, and thus the quantitatively analyzed levels of the respective proteins may be analyzed in combination to determine the occurrence of major depressive disorder.

As an example of the method for analyzing the quantitative analysis results of the respective proteins in combination, it is possible to use a method for determining the occurrence of major depressive disorder by using the quantitatively analyzed levels of the respective proteins measured from a serum sample singly or in combination.

As another example of the method for analyzing the quantitative analysis results of the respective proteins in combination, conventional statistical analysis methods may be used. Here, the statistical analysis method that can be used is not particularly limited. As an example, a linear or nonlinear regression analysis method; a linear or nonlinear classification analysis method; ANOVA; a neural network analysis method; a genetic analysis method; a support vector machine analysis method; a hierarchical cluster analysis or cluster analysis method; a hierarchical algorithm using decision trees, or Kernel principal component analysis method; a Markov Blanket analysis method; a recursive feature elimination or entropy-based recursive feature elimination analysis method; a forward floating search or backward floating search analysis method; and the like may be used singly or in combination.

The combination of the quantitative analysis results may be performed using a computer algorithm capable of automatically performing the statistical methods.

Still another embodiment of the present invention provides a composition for determining the occurrence of major depressive disorder, comprising an agent for measurement of the expression level of a marker selected from the group consisting of ZA2G (zinc-alpha-2-glycoprotein), prothrombin, and a combination thereof.

As used herein, the term “agent for measurement of the quantitatively analyzed level” refers to an agent capable of specifically binding to and recognizing the protein or mRNA encoding the same or amplifying the miRNA. As a specific example, the agent may be an antibody that specifically binds to the protein, or a primer or probe that specifically binds to the miRNA, but is not limited thereto, and those skilled in the art will be able to select an appropriate agent for the purpose of the invention.

The agent may be labeled directly or indirectly to measure the expression level of the protein or mRNA. Specifically, ligands, beads, radionuclides, enzymes, substrates, cofactors, inhibitors, fluorescers, chemiluminescent materials, magnetic particles, haptens and dyes may be used as the label, but the label is not limited thereto. As specific examples, the ligands include biotin, avidin and streptavidin, the enzymes include luciferase, peroxidase and beta galactosidase, and the fluorescers include fluorescein, coumarin, rhodamine, phycoerythrin, and sulforhodamine 101 acid chloride (Texas Red), but the label is not limited thereto. Most known labels may be used as such detectable labels, and those skilled in the art will be able to select an appropriate label for the purpose of the invention.

As used herein, the term “antibody” refers to a proteinaceous molecule capable of specifically binding to an antigenic site of a protein or peptide molecule. Such an antibody can be prepared by a conventional method from a protein to be encoded by the marker gene, the protein being obtained by cloning each gene into an expression vector according to a conventional method. The form of the antibody is not particularly limited, and a part thereof is also included in the antibody of the present invention as long as it has a polyclonal antibody, a monoclonal antibody, or antigen-binding property. Not only all immunoglobulin antibodies but also special antibodies such as humanized antibodies may be included. In addition, the antibody includes functional fragments of antibody molecules as well as complete forms having two full-length light chains and two full-length heavy chains. A functional fragment of an antibody molecule means a fragment having at least an antigen-binding function, and may be Fab, F(ab′), F(ab′)₂, and Fv.

As used herein, the term “primer” refers to a nucleotide sequence having a short tree 3′ hydroxyl group, and refers to a short sequence capable of forming a base pair with a complementary template and functioning as a starting point for template strand copying. In the present invention, the primer used for the miRNA amplification may be a single-stranded oligonucleotide that can act as a starting point for template-directed DNA synthesis in an appropriate buffer at an appropriate temperature under appropriate conditions (for example, four different nucleoside triphosphates and a polymerase such as DNA/RNA polymerase or reverse transcriptase). The appropriate length of the primer may vary depending on the purpose of use. The primer sequence is not required to be completely complementary to the polynucleotide of the miRNA of the gene or its complementary polynucleotide, and any primer can be used as long as the primer sequence is sufficiently complementary to hybridize.

As used herein, the term “probe” refers to a labeled nucleic acid fragment or peptide capable of specifically binding to miRNA. As specific examples, the probe may be constructed in the form of an oligonucleotide probe, a single stranded DNA probe, a double stranded DNA probe, an RNA probe, an oligonucleotide peptide probe, a polypeptide probe, or the like.

The composition may further comprise an agent for measurement of the expression level of K2C1 in addition to the agents for measurement of the expression levels of ZA2G and prothrombin as described above.

Still another embodiment of the present invention provides a kit for determining the occurrence of major depressive disorder, comprising quantitative devices for measurement of the expression levels of one or more proteins selected from the group consisting of ZA2G (zinc-alpha-2-glycoprotein), prothrombin, and a combination thereof.

The quantitative device included in the diagnostic kit of the present invention may measure the expression level of the marker protein. As a specific example, the quantitative device may be an RT-PCR kit or an ELISA kit, but is not limited thereto as long as the expression level of miRNA or protein can be measured.

In this case, the RT-PCR kit may be a kit including essential elements necessary to perform RT-PCR. For example, the RT-PCR kit may contain, in addition to each primer specific for the gene, a test tube or another suitable container, reaction buffers (pH and magnesium concentrations vary), deoxynucleotides (dNTPs), dideoxynucleotides (ddNTPs), enzymes such as Taq-polymerase and reverse transcriptase, DNase, RNAse inhibitors, DEPC-water, sterile water, and the like. In addition, a primer pair specific for the gene used as a quantitative control may be contained.

The kit may further comprise a quantitative device for measurement of the expression level of K2C1 in addition to the quantitative devices for measurement of the expression levels of ZA2G and prothrombin as described above.

Still another embodiment of the present invention provides the use of one or more proteins selected from the group consisting of ZA2G (zinc-alpha-2-glycoprotein), prothrombin, and a combination thereof to determine the occurrence of major depressive disorder.

### Advantageous Effects

The method for providing information for use in determining the occurrence of major depressive disorder provided by the present invention can be widely utilized to determine the occurrence of various mental disorders, including major depressive disorder since it is possible to measure the expression levels of proteins of which the expression levels are changed at the time of the occurrence of major depressive disorder, and to more objectively and accurately determine the occurrence of major depressive disorder when the method is used.

### BRIEF DESCRIPTION OF DRAWINGS

FIG. 1A is graphs illustrating the analysis results of changes in the expression levels of ZA2G and K2C1 in patients in depression status and the same patients in remission status:

FIG. 1B is graphs illustrating the comparative analysis results of changes in the expression levels of ZA2G and K2C1 in patients in depression status, the same patients in remission status, and a control group;

FIG. 2A is graphs illustrating the analysis results of changes in the expression level of prothrombin in patients in depression status and patients in remission status; and

FIG. 2B is graphs illustrating the comparative analysis results of changes in the expression level of prothrombin in patients in depression status, patients in remission status, and a control group.

### DETAILED DESCRIPTION OF THE INVENTION

Hereinafter, the configuration and effects of the present invention will be described in more detail with reference to exemplary embodiments. However, these exemplary embodiments are for illustrative purposes only, and the scope of the present invention is not intended to be limited by these exemplary embodiments.

### Example 1: Discovery of Marker for Diagnosis of Major Depressive Disorder from the Same Patient with Major Depressive Disorder

First, blood was collected from 13 patients with major depressive disorder in depression status (ADT) and the same 13 patients in remission status (ART), and sera were obtained from the blood samples. Here, ADT refers to a status in which a person has been diagnosed with major depressive disorder, has symptoms of major depressive disorder, and has undergone drug treatment for major depressive disorder, and ART refers to a status in which a patient was diagnosed with major depressive disorder, but does not currently have symptoms of major depressive disorder, and treatment for major depressive disorder has been completed.

A portion of each of the obtained sera was fractionated, and the respective fractionated sera were combined into one to obtain a pooled sample.

Each of the obtained serum samples and pooled samples was applied to a MARS (Multiple Affinity Removal System) column to deplete highly abundant proteins in the serum contained in each sample.

Each of the serum samples and pooled samples treated in the MARS column were treated with trypsin, and proteins contained in each sample were decomposed into peptides to obtain peptide serum samples and peptide pooled samples.

The OFFGEL fractionation method was performed on the peptide pooled sample. Roughly, the peptide pooled sample was electrophoresed and sorted by pl value to obtain 12 fractions.

DIA MS analysis was performed on the 12 fractionated samples thus obtained, and a peptide library was constructed based on the results.

DIA MS analysis was performed on the obtained peptide serum sample, and the MS/MS data thus acquired was applied to the peptide library constructed previously by the spectrum matching method to perform relative quantitative analysis. A targeted marker peptide candidate was selected by statistical analysis of the relative quantitative analysis data thus acquired. Here, the selection criteria were set to the following six items:

(1) there is no identical sequence in different proteins;

(2) R or K is present at one of both ends of the peptide sequence (use of trypsin);

(3) there is no M in the middle of the sequence (amino acid that is easily oxidized);

(4) the length of the peptide sequence is 15 or less;

(5) it is a sequence without post-translational modifications; and

(6) the peptide has an intensity of 1000 or more and FDR of 1 or less.

Absolute quantitative analysis using MRM (Multiple Reaction Monitoring) was performed on the targeted marker peptides selected according to the criteria.

Roughly, the concentration of the peptide was determined using the standard curve of the targeted marker peptide selected, and then it was confirmed whether the expression tendency of the targeted marker peptide derived from the result of the relative quantitative analysis performed previously was consistent with the MRM result (FIG. 1A).

FIG. 1A is graphs illustrating the analysis results of changes in the expression levels of ZA2G and K2C1 in patients in depression status and the same patients in remission status.

As illustrated in FIG. 1A, it was confirmed that the expression levels of ZA2G and K2C1 were downregulated in the patients in depression status compared to in the same patients in remission status.

In order to verify this, the expression levels of ZA2G and K2C1 in patients in depression status and in the same patients in remission status previously measured were compared with the expression levels of ZA2G and K2C1 in a control group (FIG. 1B). At this time, serum samples obtained from 67 healthy controls were used as the control group.

FIG. 1B is graphs illustrating the comparative analysis results of changes in the expression levels of ZA2G and K2C1 in patients in depression status, the same patients in remission status, and the control group.

As illustrated in FIG. 1B, it was confirmed that the expression levels of ZA2G and K2C1 were statistically significantly lower in the patients in depression status compared to the control group.

In contrast, statistically significant changes in the expression levels of ZA2G and K2C1 were not confirmed in the patients in remission status compared to in the control group.

### Example 2: Discovery of Marker for Diagnosis of Major Depressive Disorder from Patient with Major Depressive Disorder

Marker proteins for diagnosis of major depressive disorder were discovered using the method of Example 1 except that sera obtained from 22 patients in depression status and 20 patients in remission status were used instead of the sera obtained from 13 patients with major depressive disorder in depression status (ADT) and the same 13 patients in remission status (ART) (FIG. 2A).

FIG. 2A is graphs illustrating the analysis results of changes in the expression level of prothrombin in patients in depression status and patients in remission status.

As illustrated in FIG. 2A, it was confirmed that the expression level of prothrombin is statistically significantly upregulated in the patients in depression status compared to in the patients in remission status.

In order to verity this, the expression level of prothrombin was compared using serum samples obtained from 47 new patients in depression status, 40 new patients in remission status, and a control group (FIG. 2B). At this time, serum samples obtained from 67 healthy controls were used as the control group.

FIG. 2B is graphs illustrating the comparative analysis results of changes in the expression level of prothrombin in patients in depression status, patients in remission status, and the control group.

As illustrated in FIG. 2B, it was confirmed the expression level of prothrombin was statistically significantly higher in the patients in depression status than in the control group.

In contrast, the expression level of prothrombin was statistically significantly lower in the patients in remission status than in the control group.

Based on the above description, it will be understood by those skilled in the art that the present disclosure may be implemented in a different specific form without changing the technical spirit or essential characteristics thereof. Therefore, it should be understood that the above embodiment is not limitative, but illustrative in all aspects. The scope of the disclosure is defined by the appended claims rather than by the description preceding them, and therefore all changes and modifications that fall within metes and bounds of the claims or equivalents of such metes and bounds are therefore intended to be embraced by the claims.

1. A method for providing information necessary to determine occurrence of major depressive disorder, the method comprising:
(a) quantitatively analyzing an expression level of a marker protein selected from the group consisting of ZA2G (zinc-alpha-2-glycoprotein), prothrombin, and a combination thereof in a serum sample of an individual suspected of having major depressive disorder; and
(b) correlating the quantitatively analyzed expression level of the marker protein with determination of occurrence of major depressive disorder.

2. The method according to claim 1, wherein the step (b) is performed by combining quantitative analysis results of respective marker proteins.

3. The method according to claim 2, wherein the combining is performed using an analysis method selected from the group consisting of a linear or nonlinear regression analysis method; a linear or nonlinear classification analysis method; ANOVA; a neural network analysis method; a genetic analysis method; a support vector machine analysis method; a hierarchical cluster analysis or cluster analysis method; a hierarchical algorithm using decision trees, or Kernel principal component analysis method; a Markov Blanket analysis method; a recursive feature elimination or entropy-based recursive feature elimination analysis method; a forward floating search or backward floating search analysis method; and a combination thereof.

4. The method according to claim 2, wherein the combining is performed using a computer algorithm.

5. The method according to claim 1, wherein the step (a) further includes quantitatively analyzing an expression level of K2C1 (keratin type II, cytoskeletal 1).

6. The method according to claim 1, wherein the step (b) further includes correlating a quantitatively analyzed expression level of K2C1 with determination of occurrence of major depressive disorder.

7. The method according to claim 6, wherein the correlating step is performed by combining quantitative analysis results of ZA2G, prothrombin, and K2C1.
